SEQUENCE LISTING (11 sequences)

SEQ ID NO: 1 - DNA, 1275 bp, Thermococcus litoralis
atggaatttaacaaagttttttctctgctgttggtctttgttgtacttggagctacagcggggatagtaggggcagtgtctgccgagaaagttcgggtgataataacaatagacaaggactttaacgaaaactccgtctttgcacttggaggaaacgttgttgcaagaggaaaggtatttccaatcgttatagcggagctttctccacgagcagttgaaaggctaaagaatgctaagggtgtcgtgagagtagagtacgatgcagaagtgcaggtattaaagggcaaatccccgggagcaggcaagccaaagccttcacaaccagctcaaacgattccatggggaattgaaaggattaaagccccggatgtatggagcataactgacggttcaagtagtggagtaattgaggttgcaatcctagatactggaattgattatgaccatccagatttagcggcaaatctcgcgtggggtgtaagcgtacttaggggcaaagtgtccacaaagcccaaagattacaaagaccagaatggccatgggactcatgttgcgggaactgtagcggcactcaataatgacattggagttgtaggagtcgccccagctgtggagatctatgctgttagggttcttgatgcaagcggtagaggatcctatagcgacataatccttggaatagagcaagcactgcttggtcccgatggagttcttgacagtgacggagatggaataatagtgggtgatccggatgatgatgcggccgaagtcataagcatgagccttggaggtttaagcgatgttcaagccttccatgatgcaataatagaggcatacaattacggagtagtcattgtggcggcaagtggtaatgagggagcctcaagcccaagctatccagcagcttatccggaggttatagccgttggggcaactgacgttaatgatcaagtaccttggtggagcaacaggggagtggaagtaagtgctcctggcgttgatgtactaagcacgtatccggacgatagttatgagacgcttagcggcacttcaatggcaacaccccatgtaagcggagttgtggcgctaatccaagcggcgtactacaacaaatatggaagtgttcttccggttggaacgtttgatgataataccatgagcactgttaggggaattctacacatcacggctgacgaccttggaagctcgggttgggatgcagactatggttatggaatagttagagcggatttagctgttcaagctgtcaactga

SEQ ID NO: 2 - protein, 424 aa, Thermococcus litoralis
MEFNKVFSLLLVFVVLGATAGIVGAVSAEKVRVIITIDKDFNENSVFALGGNVVARGKVFPIVIAELSPRAVERLKNAKGVVRVEYDAEVQVLKGKSPGAGKPKPSQPAQTIPWGIERIKAPDVWSITDGSSSGVIEVAILDTGIDYDHPDLAANLAWGVSVLRGKVSTKPKDYKDQNGHGTHVAGTVAALNNDIGVVGVAPAVEIYAVRVLDASGRGSYSDIILGIEQALLGPDGVLDSDGDGIIVGDPDDDAAEVISMSLGGLSDVQAFHDAIIEAYNYGVVIVAASGNEGASSPSYPAAYPEVIAVGATDVNDQVPWWSNRGVEVSAPGVDVLSTYPDDSYETLSGTSMATPHVSGVVALIQAAYYNKYGSVLPVGTFDDNTMSTVRGILHITADDLGSSGWDADYGYGIVRADLAVQAVN

SEQ ID NO: 3 - DNA, 1281 bp, Artificial
  Expression construct including Savinase signal peptid instead of native signal
atgaaaaaaccgctggggaaaattgtcgcaagcaccgcactactcatttctgttgcttttagttcatcgatcgcatcggctgtgtctgccgagaaagttcgggtgataataacaatagacaaggactttaacgaaaactccgtctttgcacttggaggaaacgttgttgcaagaggaaaggtatttccaatcgttatagcggagctttctccacgagcagttgaaaggctaaagaatgctaagggtgtcgtgagagtagagtacgatgcagaagtgcaggtattaaagggcaaatccccgggagcaggcaagccaaagccttcacaaccagctcaaacgattccatggggaattgaaaggattaaagccccggatgtatggagcataactgacggttcaagtagtggagtaattgaggttgcaatcctagatactggaattgattatgaccatccagatttagcggcaaatctcgcgtggggtgtaagcgtacttaggggcaaagtgtccacaaagcccaaagattacaaagaccagaatggccatgggactcatgttgcgggaactgtagcggcactcaataatgacattggagttgtaggagtcgccccagctgtggagatctatgctgttagggttcttgatgcaagcggtagaggatcctatagcgacataatccttggaatagagcaagcactgcttggtcccgatggagttcttgacagtgacggagatggaataatagtgggtgatccggatgatgatgcggccgaagtcataagcatgagccttggaggtttaagcgatgttcaagccttccatgatgcaataatagaggcatacaattacggagtagtcattgtggcggcaagtggtaatgagggagcctcaagcccaagctatccagcagcttatccggaggttatagccgttggggcaactgacgttaatgatcaagtaccttggtggagcaacaggggagtggaagtaagtgctcctggcgttgatgtactaagcacgtatccggacgatagttatgagacgcttagcggcacttcaatggcaacaccccatgtaagcggagttgtggcgctaatccaagcggcgtactacaacaaatatggaagtgttcttccggttggaacgtttgatgataataccatgagcactgttaggggaattctacacatcacggctgacgaccttggaagctcgggttgggatgcagactatggttatggaatagttagagcggatttagctgttcaagctgtcaactga

SEQ ID NO: 4 - protein, 27 aa, Bacillus clausii
MKKPLGKIVASTALLISVAFSSSIASA

SEQ ID NO: 5 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 6 - protein, 583 aa, Artificial
  Hybrid alpha-amylase
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 7 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 8 - DNA, 1278 bp, Thermococcus sp
atggaatttaacaaagttttttctctgctgttggtctttgttgtacttggagctacagcagggatagtaggggcagcgcctgctgagaaagctcgagtgataataacaatagacaaggactttaacgaaaactccgtctttgcacttggaggcaacgttgttgcaagaggaaaggtatttccaattgttatagcggagcttcctccacgagcaattgagagattaaagaatgctaagggtgttgttagagtagaatacgatgcggaggcccatatattaaaaggcaaaccaccgggaacaggcaagccaaagccttcacaaccagctcaaacgattccatggggaattgaaaggattaaagccccggatgcatggagcataactgatggttcaagtggtggagtaattgaggttgcaatcctcgatacgggaattgattatgaccatccagatttagcggcaaatctcgcgtggggtgtaagcgtacttagaggcaaagtgtctacaaatcccaaagattacaaagaccagaatggccatgggactcatgttgcgggaactgtagcggcactcaataatgacattggagtagtgggagtcgcttcagctgtggagatttatgctgttagggttcttgatgcaagtggtagaggatcttatagcgacataatccttggaatagagcaggcattgcttggccctgatggagtgcttgactccgataatgatggtgtaatagtgggagatccggacgatgatgcagctgaagtcataagcatgagccttggaggttcaagcgatgttcaagccttccatgatgcaataatagaggcatacaattacggagttgtcatcgtagcggcaagtggtaatgatggggcatcaagtccaagttaccctgcagcttatccagaggttatagccgttggtgcaacagatagcgatgaccaagtaccttggtggagcaacaggggagtagaagttagtgctcctggcgttgatatactaagcacgtatccggacgatacctatgaaacacttagcggcacttcaatggcaacacctcacgttagtggagtagtggcattaatccaggcggcgtactacaacaaatatgggtatgtccttccagttggaacatttggcgatcttaccacgagtactgttagggggattctacacgtaacagctgatgaccttggaagctcgggttgggatgcagactatggctatggaatagttagggcagatttggctgttcaagctgctatcagttga

SEQ ID NO: 9 - protein, 425 aa, Thermococcus sp
MEFNKVFSLLLVFVVLGATAGIVGAAPAEKARVIITIDKDFNENSVFALGGNVVARGKVFPIVIAELPPRAIERLKNAKGVVRVEYDAEAHILKGKPPGTGKPKPSQPAQTIPWGIERIKAPDAWSITDGSSGGVIEVAILDTGIDYDHPDLAANLAWGVSVLRGKVSTNPKDYKDQNGHGTHVAGTVAALNNDIGVVGVASAVEIYAVRVLDASGRGSYSDIILGIEQALLGPDGVLDSDNDGVIVGDPDDDAAEVISMSLGGSSDVQAFHDAIIEAYNYGVVIVAASGNDGASSPSYPAAYPEVIAVGATDSDDQVPWWSNRGVEVSAPGVDILSTYPDDTYETLSGTSMATPHVSGVVALIQAAYYNKYGYVLPVGTFGDLTTSTVRGILHVTADDLGSSGWDADYGYGIVRADLAVQAAIS

SEQ ID NO: 10 - DNA, 1284 bp, Artificial
  Expression construct including Savinase signal peptid instead of native signal
atgaagaaaccgttggggaaaattgtcgcaagcaccgcactactcatttctgttgcttttagttcatcgatagcatcagcagcaccagcagagaaggcacgtgttatcatcactattgataaggactttaacgagaattcagttttcgctttaggtggtaatgtagttgctcgcggaaaagttttccctattgttatcgcggaacttcctcctcgtgcaatcgaacgtttgaaaaacgctaaaggcgtagttcgtgttgaatacgatgcggaagctcacatccttaaaggcaaacctccgggtactggtaagccaaaaccgtctcaaccggctcaaactatcccgtggggtatcgaacgtatcaaagcaccggacgcatggtctattacagacggctcttctggtggtgtaattgaagtagcgatcttagatacaggaatcgactacgatcatcctgatcttgcagcgaacttggcatggggtgtatctgttcttcgtggtaaagtatctactaaccctaaagactacaaggaccaaaacggacacggtacgcatgttgcaggaactgtagcagcgttgaacaacgacattggagttgttggcgtagcgtctgctgtagagatctatgctgttcgtgttcttgatgcgtctggtcgtggaagctattctgacatcattcttggaatcgaacaagcattacttggtcctgacggagttttggattcagataacgatggtgttatcgtaggtgaccctgatgacgacgctgctgaagttatctcaatgagccttggtggctcttcagacgttcaagccttccatgacgcaatcatcgaagcttacaactatggagttgttattgttgcggcatctggaaacgacggtgcgtcaagcccttcttacccagctgcttaccctgaggtaattgctgttggagcgacagattcagatgaccaggtaccttggtggtcaaatcgcggtgttgaagtttctgctcctggagttgatatccttagcacataccctgatgacacttacgaaacactttcaggcacttctatggctacacctcatgtttctggcgtagtagctcttatccaagctgcgtattacaacaaatacggttatgttcttccagttggcacatttggagatcttacgacgagcacggttcgcggtattcttcatgttacagcggacgacttaggctcttctggctgggatgctgattatggttacggtattgtacgtgctgacttagcagttcaggcagcaatcagctaa

SEQ ID NO: 11 - protein, 366 aa, Meripilus giganteus [sequence omitted]

(19) United States
(12) Patent Application Publication
(10) Pub. No.: US 2018/0327711 A1
(43) Pub. Date: Nov. 15, 2018

(54) Fermentation Processes With Reduced Foaming

(71) Applicant: Novozymes A/S, Bagsvaerd (DK)

(72) Inventors: Felipe Senne De Oliveira Lino, Paraná (BR); Camila Do Nascimento, Paraná (BR); João G. R. Destro, Paraná (BR); Kenneth Jensen, Bagsvaerd (DK)

(73) Assignee: Novozymes A/S, Bagsvaerd (DK)

(21) Appl. No.: 15/774,737

(22) PCT Filed: Nov. 11, 2016
(86) PCT No.: PCT/EP2016/077467

§ 371 Date: May 9, 2018

(30) Foreign Application Priority Data
Nov. 12, 2015 (EP) 15194234.9
Nov. 12, 2015 (EP) 15194235.6

(51) Int. Cl. C12N 1/34; C12P 7/06; C12N 1/18

(52) U.S. Cl. CPC C12N 1/34; C12N 1/18; C12P 7/06

(57) ABSTRACT

The present invention relates to processes of producing a fermentation product from readily fermentable sugar-material in a fermentation vat comprising a fermentation medium, comprising: feeding the readily fermentable sugar-material into the fermentation vat comprising a slurry of fermenting organism; fermenting the readily fermentable sugar material into a desired fermentation product, wherein S8A protease is added during or after feeding of the readily fermentable sugar-material into fermentation vat or during fermentation of the readily fermentable sugar-material into the desired fermentation product. The invention also related to the use of S8A protease for reducing foaming in the fermentation wells generating by the fermenting organism during fermentation of the readily fermentable sugar-material.

### FIELD OF THE INVENTION

The present invention relates to reducing foaming in fermentation processes for producing fermentation products, such as ethanol, from readily fermentable sugar materials.

### REFERENCE TO A SEQUENCE LISTING

This application contains a Sequence Listing in computer readable form. The computer readable form is incorporated herein by reference.

### BACKGROUND OF THE INVENTION

Fermentation products, such as ethanol, can be produced from a wide range of renewable feedstocks. These can be classified in three main groups: (1) readily fermentable sugar materials, such as sugar cane (i.e., sugar cane juice and molasses), sugar beets, sweet sorghum; (2) starchy materials, such as corn, potatoes, rice, wheat, agave; and (3) cellulosic materials, such as stover, grasses, corn cobs, wood and sugar cane bagasse. The readily fermentable sugar material contains simple sugars, such as sucrose, glucose and fructose, that can readily be fermented by yeast.

Readily fermentable sugar materials, such as sugar cane juice and molasses, are used as substrates in, e.g., Brazilian ethanol production. Yeast, such as especially Saccharomyces cerevisiae, is used as the fermentation organism. Often a yeast recycling system is used where up to 90-95% of the yeast is reused from one fermentation cycle to the next. This results in very high cell densities inside the fermentation vat (e.g., 8-17% w/v, wet basis) and in a very short fermentation time. Ethanol concentrations of 8-11% (v/v) are achieved within a period of 6-11 hours at around 32° C. After every batch fermentation, yeast cells are collected by centrifugation, acid washed (e.g., sulfuric acid at pH 1.5-3.0 for 1-2 hours) and sent back to the fermentation vat. Today a chemical defoamer (dispersant) is added during acid wash at a fixed dosage after each cycle and another chemical defoamer (antifoam) is added directly into the fermentation vat automatically (when foam reaches a level sensor) or manually until foam is fully controlled.

U.S. Pat. No. 3,959,175 discloses an aqueous defoamer composition containing liquid polybutene. The defoamer composition can further comprise in part hydrophobic silica and silicone oils.

U.S. Pat. No. 5,288,789 discloses the use of a condensate of alkylphenol and aldehyde that has been polyoxyalkylated to reduce foam in a fermentation broth.

U.S. Pat. No. 6,083,998 concerns defoamer compositions for alcoholic fermentations which as aqueous based and comprise polydimethylsiloxane oils, ethylene oxide/propylene oxide block copolymers and a silicone/silica blend.

When producing ethanol from readily fermentable sugar materials, such as sugar cane juice and molasses, foam generated by the fermenting organism is a serious problem.

Even though chemical defoamers can be used there is still a desire and need for providing processes for producing fermentation products, such as ethanol, where the foam generation is reduced/controlled.

WO 2014/205198 discloses protease from Pyrocuccus furiosus which can reduce foam generated by fermenting organisms during fermentation when producing fermentation products, such as especially ethanol, from readily fermentable sugar materials, such as sugar cane molasses.

The present invention provides S8A proteases which demonstrates better performance in foam reduction compared to protease from Pyrocuccus furiosus, which is an intracellular enzyme and expensive to be used in fermentation process.

### SUMMARY OF THE INVENTION

When producing fermentation products, such as especially ethanol, from readily fermentable sugar-materials, such as sugar cane juice and molasses, foam generated by the fermenting organism is a serious problem. Thus, the object of the present invention is to reduce foam generated by fermenting organisms during fermentation when producing fermentation products, such as especially ethanol, from readily fermentable sugar materials, such as sugar cane molasses. The inventors surprisingly found that Thermococcus sp S8A proteases can be used to effectively solve the foaming problem.

A first aspect of the invention relates to a process of producing a fermentation product from readily fermentable sugar-material in a fermentation vat comprising a fermentation medium using a fermenting organism, comprising:

i) feeding the readily fermentable sugar-material into the fermentation vat comprising a slurry of fermenting organism;

ii) fermenting the readily fermentable sugar-material into a desired fermentation product,

wherein a Thermococcus sp S8A protease is added

a) before, during and/or after feeding in step i), and/or

b) during fermentation in step ii).

In a second aspect the invention relates to use of Thermococcus sp. S8A proteases for reducing foam generated by fermenting organisms when producing a desired fermentation product from readily fermentable sugars.

### Definitions

S8A Protease: The term “S8A protease” means an S8 protease belonging to subfamily A. Subtilisins, EC 3.4.21.62, are a subgroup in subfamily S8A, however, the present S8A proteases from Thermococcus litoralis or Thermococcus sp PK are subtilisin-like proteases, which have not yet been included in the IUBMB classification system. The S8A protease according to the invention hydrolyses the substrate Suc-Ala-Ala-Pro-Phe-pNA. The release of p-nitroaniline (pNA) results in an increase of absorbance at 405 nm and is proportional to the enzyme activity. pH optimum=pH 8, and Temperature optimum=60° C.

In one aspect, the polypeptides of the present invention have at least 20%, e.g., at least 40%, at least 50%, at least 60%, at least 70%, at least 80%, at least 90%, at least 95%, or at least 100% of the protease activity of the mature polypeptide of SEQ ID NO: 2. In another aspect, the S8A protease has at least 20%, e.g., at least 40%, at least 50%, at least 60%, at least 70%, at least 80%, at least 90%, at least 95%, or at least 100% of the protease activity of the mature polypeptide of SEQ ID NO: 9.

In one embodiment protease activity can be determined by the kinetic Suc-AAPF-pNA assay as disclosed herein and as exemplified in example 6 and 8.

Fragment: The term “fragment” means a polypeptide having one or more (e.g., several) amino acids absent from the amino and/or carboxyl terminus of a mature polypeptide or domain; wherein the fragment has protease activity. In one aspect, a fragment contains at least 314 amino acid residues (e.g., amino acids 111 to 424 of SEQ ID NO: 2, particularly amino acids 110 to 424, more particularly amino acids 109 to 424, more particularly amino acids 108 to 424, even more particularly amino acids 107 to 424 of SEQ ID NO: 2). In another embodiment a fragment contains at least 315 amino acid residues (e.g., amino acids 111 to 425 of SEQ ID NO: 9, particularly amino acids 110 to 425, more particularly amino acids 109 to 425, more particularly amino acids 108 to 425, even more particularly amino acids 107 to 425 of SEQ ID NO: 9).

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 107 to 424 of SEQ ID NO: 2. Amino acids 1 to 25 of SEQ ID NO: 2 are a signal peptide. Amino acids 26 to 106 are a pro-peptide. In another aspect, the mature polypeptide is from amino acids 107 to 425, particularly from amino acids 108-425 and more particularly from amino acids 109-425 of SEQ ID NO: 9. Amino acids 1 to 25 of SEQ ID NO: 9 are a signal peptide. Amino acids 26 to 106, particularly amino acids 26-107 and more particularly amino acids 26-108 are a pro-peptide. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide. The N-terminal was confirmed by MS-EDMAN data on the purified protease as shown in the examples section.

Mature polypeptide coding sequence: The term “mature polypeptide coding sequence” means a polynucleotide that encodes a mature polypeptide having protease activity. In one aspect, the mature polypeptide coding sequence is nucleotides 319 to 1272 of SEQ ID NO: 1, nucleotides 76 to 318 encode a propeptide, and nucleotides 1 to 75 of SEQ ID NO: 1 encode a signal peptide. In another aspect, the mature polypeptide coding sequence is nucleotides 319 to 1275, or nucleotides 322 to 1275, or nucleotides 325 to 1275 of SEQ ID NO: 1, nucleotides 76 to 318, or nucleotides 76 to 321, or nucleotides 76 to 324 encode a propeptide, and nucleotides 1 to 75 of SEQ ID NO: 8 encode a signal peptide.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.]

Subsequence: The term “subsequence” means a polynucleotide having one or more (e.g., several) nucleotides absent from the 5′ and/or 3′ end of a mature polypeptide coding sequence; wherein the subsequence encodes a fragment having protease activity.

Variant: The term “variant” means a polypeptide having protease activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In describing variants, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The object of the present invention is to reduce foaming generated by fermenting organisms, especially foaming yeast, such as of the genus Saccharomyces, in particular Saccharomyces cerevisae yeast, during fermentation when producing a desired fermentation product, such as especially ethanol, from readily fermentable sugar material, such as especially sugar cane molasses. In a preferred embodiment the invention relates to a Brazilian-type ethanol fermentation process, e.g., as describe by Basso et al in (2011) in “Ethanol Production in Brazil: The Industrial Process and Its Impact on Yeast Fermentation, Biofuel Production-Recent Developments and Prospects, Dr. Marco Aurelio Dos Santos Bernardes (Ed.), ISBN: 978-953-307-478-8, InTech.” Generally Brazilian ethanol processes include recycling of the fermenting organisms, especially foaming fermenting yeast, such as Saccharomyces cerevisae yeast, and are carried out as batch or fed batch processes. However, some plants do semi-continuous and continuous fermentation processes.

The inventors have found a number of surprising advantages of adding Thermococcus sp. S8A proteases in with a process of the invention.

In WO2014/205198 it was disclosed that a serine protease from Pyrococcus furiosus can be used efficiently instead of chemicals for reducing foaming when producing ethanol from readily fermentable sugar materials such as sugar cane molasses. However, since PfuS is a difficult protease to express, due to intracellular expression, alternative proteases are desirable.

Thus in a first aspect the present invention relates to a process of producing a fermentation product from readily fermentable sugar-material in a fermentation vat comprising a fermentation medium using a fermenting organism, comprising:

i) feeding the readily fermentable sugar material into the fermentation vat comprising a slurry of fermenting organism;
ii) fermenting the readily fermentable sugar material into a desired fermentation product,
wherein a Thermococcus species S8A protease is added
a) before, during and/or after feeding in step i), and/or
b) during fermentation in step ii).

According to the invention the term “readily fermentable sugar-material” means that the sugar-containing starting material to be converted/fermented into a desired fermentation product, such as especially ethanol, is of the kind which contains simple sugars, such as sucrose, glucose and fructose, that can be readily fermented by the fermenting organism, such as especially yeast strains derived from Saccharomyces cerevisae.

According to the invention the term “fermentation vat” means and includes any type of fermentation vat, fermentation vessel, fermentation tank, or fermentation container, or the like, in which fermentation is carried out.

According to the invention in steps i) and ii) may be carried out simultaneously or sequentially. The fermentation may be carried out at a temperature from 25° C. to 40° C., such as from 28° C. to 35° C., such as from 30° C. to 34° C., preferably around 32° C. In an embodiment the fermentation is ongoing for 2 to 120 hours, in particular 4 to 96 hours. In an embodiment the fermentation may be done in less than 24 hours, such as less than 12 hours, such as between 6 and 12 hours.

In contrast to starch-containing feedstocks, such as corn, wheat, rye, milo, sorghum etc., and cellulosic feedstocks, such corn cobs, corn stover, bagasse, wheat straw, wood etc. there is no need for pretreatment and/or (prior) hydrolysis before fermentation. In a preferred embodiment the readily fermentable sugar-material is selected from the group consisting of sugar cane juice, sugar cane molasses, sweet sorghum, sugar beets, and mixture thereof. However, according to the invention the fermentation medium may also further comprise other by-products of sugar cane, in particular hydrolysate from sugar cane bagasse. In an embodiment the fermentation medium may include separate streams comprising, e.g., C5-liquor, etc. According to the invention the readily fermentable sugar-material (substrate) does not include a substantial content of polysaccharide, such as starch and/or cellulose/hemicellulose.

In a preferred embodiment the fermenting organism used in a process of the invention may be a foaming fermenting organism capable of fermenting readily fermentable sugar-material into a desired fermentation product, such as especially ethanol. Many commercial yeast strains, including especially strains of Saccharomyces cerevisae, used commercially, e.g., in Brazil, today, e.g., for producing ethanol from sugar cane molasses generate foam during fermentation. In an embodiment the fermenting organism is a yeast, e.g., from a strain of the genus Saccharomyces, such as a strain of Saccharomyces cerevisiae. Thus, in a preferred embodiment the fermenting organism is a foaming fermenting organism, such as a foaming strain of Saccharomyces, such as especially a strain of Saccharomyces cerevisae generating foam during fermentation. According to the invention the density of yeast in the fermentation medium is high, such as from 8-17% w/v, wet basis of the fermentation medium. In an embodiment, the fermentation occurs at non-aseptically conditions, e.g., where wild yeast strains with a foaming phenotype may also be introduced to the fermentation vat and incorporated into the yeast population.

In a preferred embodiment of the invention the fermenting organisms are recycled after fermentation in step ii). According to the invention from 50-100%, such as 70-95%, such as about 90% of the fermentation organisms are recycled. The fermenting organisms, such as yeast, are collected after fermentation in step ii), acid washed, and recycled to the fermentation vat. The fermenting organisms are acid washed with sulfuric acid, e.g., at pH 1.5-3.0, such as 2.0-2.5, e.g., for 1-2 hours. The process of the invention may be carried out as a batch or fed-batch fermentation. However, the process of the invention may also be done as a semi-continuous or continuous process.

The terms “fermentation product” and “desired fermentation product” mean a product produced by fermentation using a fermenting organism. Fermentation products contemplated according to the invention include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H2 and CO2); antibiotics (e.g., penicillin and tetracycline); enzymes; vitamins (e.g., riboflavin, B12, beta-carotene); and hormones.

In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. According to the invention the preferred fermentation product is ethanol. The desired fermentation product, such as ethanol, obtained according to the invention, may preferably be used as fuel, e.g., for vehicles, such as cars. Fuel ethanol may be blended with gasoline. Ethanol it may also be used as potable ethanol.

Subsequent to fermentation in step ii) the desired fermentation product, such as ethanol, may be separated from the fermentation medium, e.g., by distillation, or another separation technology. Alternatively, the desired fermentation product may be extracted from the fermentation medium by micro or membrane filtration techniques. The fermentation product may also be recovered by stripping or other method well-known in the art.

In one embodiment the readily fermentable sugar material is feed into the fermentation vat as a feeding stream. It is contemplated that the Thermococcus S8A protease may be added before or during feeding of the readily fermentable sugar material. Thus in one embodiment the Thermococcus sp. S8A protease is mixed with the feeding stream of the readily fermentable sugar-material. In another embodiment the Thermococcus sp. S8A protease is mixed with the feeding stream of readily fermentable sugar-material before feeding step i).

### Specific Embodiments of the Process of the Invention

In one embodiment of the process of the invention, the desired fermentation product is produced from readily fermentable sugar material by fermentation in a fermentation vat, the process comprises adding Thermococcus sp. S8A protease to the readily fermentable sugar material before feeding; feeding the protease-containing readily fermentable sugar material into the fermentation vat comprising a slurry of fermenting organisms; fermenting the readily fermentable sugar material into the desired fermentation product.

In another embodiment of the process of the invention, ethanol is produced in a batch, fed batch, semi-continuous or continuous fermentation process in a fermentation vat comprising sugar cane molasses, comprising adding protease to the sugar cane molasses before feeding; feeding the Thermococcus sp. S8A protease-containing sugar cane molasses into the fermentation vat comprising a slurry of Saccharomyces cerevisae yeast; and fermenting the sugar cane molasses into ethanol.

In another embodiment of the process of the invention, the desired fermentation product is produced from readily fermentable sugar material by fermentation in a fermentation vat, wherein the process comprises: feeding readily fermentable sugar material into the fermentation vat comprising a slurry of fermenting organisms; feeding Thermococcus sp. S8A protease into the fermentation vat comprising a slurry of readily fermentable sugars and fermenting organisms before fermentation; fermenting the readily fermentable sugar material into the desired fermentation product.

In another embodiment of the process of the invention, ethanol is produced in a batch or fed batch fermentation process in a fermentation vat comprising sugar cane molasses, wherein the process comprises: feeding sugar cane molasses into the fermentation vat comprising a slurry of Saccharomyces cerevisae yeast; feeding Thermococcus sp. S8A protease into the fermentation vat comprising a slurry of Saccharomyces cerevisae yeast and the sugar cane molasses before fermentation; fermenting the sugar cane molasses into ethanol.

In another embodiment of the process of the invention, the desired fermentation product is produced from readily fermentable sugar material by fermentation in a fermentation vat, wherein the process comprises: feeding readily fermentable sugar material into the fermentation vat comprising a slurry of fermenting organisms; adding Thermococcus sp. S8A protease into the fermentation vat during fermentation of the readily fermentable sugar-material into the desired fermentation product.

In another embodiment of the process of the invention, ethanol is produced as a batch, fed batch, semi-continuous or continuous fermentation process in a fermentation vat comprising sugar cane molasses, wherein the process comprises: feeding sugar cane molasses into the fermentation vat comprising a slurry of Saccharomyces cerevisae yeast; adding Thermococcus sp. S8A protease, into the fermentation vat during fermentation of the sugar cane molasses into ethanol.

In a particular embodiment the present invention relates to a process of producing a fermentation product from readily fermentable sugar-material in a fermentation vat comprising a fermentation medium using a fermenting organism, comprising:

i) feeding the readily fermentable sugar material into the fermentation vat comprising a slurry of fermenting organism;
ii) fermenting the readily fermentable sugar material into a desired fermentation product,
wherein feeding of the readily fermentable sugar-material is done by introducing a feeding stream into the fermentation vat; wherein
Thermococcus sp. S8A protease is mixed with the feeding stream before in step i); or
Thermococcus sp. S8A protease is added to fermentation vat after feeding.

In a most particular embodiment of the invention the Thermococcus sp. S8A protease is a S8A Thermococcus litoralis protease, particularly the protease disclosed as SEQ ID NO: 2, more particularly amino acids 107 to 424 of SEQ ID NO: 2. In another particular embodiment of the invention the Thermococcus sp. S8A protease is a S8A Thermococcus sp. PK protease, particularly the protease disclosed as SEQ ID NO: 9, more particularly amino acids 107 to 425 of SEQ ID NO: 9.

Another aspect of the invention relates to a use of a Thermococcus sp. S8A protease for reducing foam generated by fermenting organisms when producing a desired fermentation product from readily fermentable sugars.

A process of the invention, as defined above, includes addition of a S8A protease. In an embodiment, the present disclosure relates to S8A Thermococcus sp. protease, which is S8A Thermococcus litoralis protease or which is S8A Thermococcus PK protease. According to an embodiment of the invention the protease may, e.g., be added in a dosage from 0.2 to 25 mg Enzyme Protein (EP)/L fermentation medium.

In an embodiment the protease may be added in dosages from 0.01-100 ppm EP (Enzyme Protein) protease, such as 0.1-50 ppm, such as 1-25 ppm.

The protease may in one embodiment be the only enzyme added (i.e., no other enzymes added).

In one embodiment the S8A Thermococcus sp. protease has at least 80%, more preferably at least 85%, more preferably at least 90%, more preferably at least 91%, more preferably at least 92%, even more preferably at least 93%, most preferably at least 94%, or and even most preferably at least 95%, such as even at least 96%, at least 97%, at least 98%, at least 99%, or 100% identity to the mature part of the polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 75% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 80% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 85% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 90% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 95% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 96% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 97% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 98% of the protease activity of the mature polypeptide of SEQ ID NO: 2.

In an embodiment the S8A Thermococcus sp. protease is one having a sequence identity to the mature polypeptide of SEQ ID NO: 2 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 99% of the protease activity of the mature polypeptide of SEQ ID NO: 2.

In one embodiment the S8A Thermococcus sp. protease has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, or 100% identity to the mature part of the polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 75% of the protease activity of the mature polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 80% of the protease activity of the mature polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least 85% of the protease activity of the mature polypeptide of SEQ ID NO: 9.

In an embodiment the S8A Thermococcus sp. protease is one having a sequence identity to the mature polypeptide of SEQ ID NO: 9 having at least 80%, at least 85, at least 90%, at least 91%, at least 92%, at least 93%, at least 94%, of at least 95%, at least 96%, at least 97%, at least 98%, at least 99%, or 100%, and wherein the polypeptide has at least 90% of the protease activity of the mature polypeptide of SEQ ID NO: 9.

In an embodiment the S8A Thermococcus sp. protease is one having a sequence identity to the mature polypeptide of SEQ ID NO: 9 having at least 80%, at least 85, at least 90%, at least 91%, at least 92%, at least 93%, at least 94%, of at least 95%, at least 96%, at least 97%, at least 98%, at least 99%, or 100%, and wherein the polypeptide has at least 95% of the protease activity of the mature polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 96% of the protease activity of the mature polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 97% of the protease activity of the mature polypeptide of SEQ ID NO: 9.

In an embodiment the S8A Thermococcus sp. protease is one having a sequence identity to the mature polypeptide of SEQ ID NO: 9 having at least 80%, at least 85, at least 90%, at least 91%, at least 92%, at least 93%, at least 94%, of at least 95%, at least 96%, at least 97%, at least 98%, at least 99%, or 100%, and wherein the polypeptide has at least at least 98% of the protease activity of the mature polypeptide of SEQ ID NO: 9.

In an embodiment the S8A Thermococcus sp. protease is one having a sequence identity to the mature polypeptide of SEQ ID NO: 9 of having at least 80%, at least 85, at least 90%, at least 91%, at least 92%, at least 93%, at least 94%, at least 95%, at least 96%, at least 97%, at least 98%, at least 99%, or 100%, and wherein the polypeptide has at least at least 99% of the protease activity of the mature polypeptide of SEQ ID NO: 9.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Protease Activity

A polypeptide having protease activity of the present invention may be obtained from microorganisms of the genus Thermococcus.

In another aspect, the polypeptide is a Thermococcus litoralis polypeptide. In another aspect, the polypeptide is a Thermococcus sp. PK polypeptide.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Other Enzymes

In an embodiment the S8A protease is added together with (simultaneously with) one or more enzymes selected from the group consisting of: cellulase, glucoamylase, alpha-amylase, oxidase, peroxidase, catalase, laccase, beta-glucosidase, mannanase, other carbohydrases.

In an embodiment the S8A protease is added before and/or after the other enzymes.

According to the process of the invention adding a S8A protease results in increased yields, e.g., ethanol yield, compared to a corresponding process where no protease is present or added. The process of the invention may also reduce the residual sugars present in the fermentation medium. However, most importantly, foaming in the fermentation vat is reduced compared to a corresponding process where no S8A protease is added.

According to the invention an alpha-amylase may be added together with the protease or present and/or added during fermentation. The alpha-amylase may be of microbial origin, e.g., fungal or bacterial origin. In an embodiment the alpha-amylase is of fungal origin.

Preferably the acid fungal alpha-amylase is derived from the genus Aspergillus, especially a strain of A. terreus, A. niger, A. oryzae, A. awamori, or Aspergillus kawachii, or from the genus Rhizomucor, preferably a strain the Rhizomucor pusillus, or the genus Meripilus, preferably a strain of Meripilus giganteus.

In a preferred embodiment the alpha-amylase is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-binding domain, such as the one shown in SEQ ID NO: 6 herein, or a variant thereof.

In an embodiment the alpha-amylase is selected from the group consisting of:

(i) an alpha-amylase comprising the polypeptide of SEQ ID NO: 6 herein;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6 herein.

In a preferred embodiment the alpha-amylase is a variant of the alpha-amylase shown in SEQ ID NO: 6 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 6 for numbering).

In an embodiment the alpha-amylase is derived from a Rhizomucor pusillus with an Aspergillus niger glucoamylase linker and starch-binding domain (SBD), preferably disclosed as SEQ ID NO: 6 herein, preferably having one or more of the following substitutions: G128D, D143N, preferably G128D+D143N (using SEQ ID NO: 6 for numbering), and wherein the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6 herein.

In another embodiment the alpha-amylase may be of bacterial origin. In a preferred embodiment the bacterial alpha-amylase may be derived from the genus Bacillus, such as a strain of the species Bacillus stearothermophilus or variant thereof. The alpha-amylase may be a Bacillus stearothermophilus alpha-amylase, e.g., the mature part of the one shown in SEQ ID NO: 5 herein, or a mature alpha-amylase or a corresponding mature alpha-amylase having at least 60%, such as 70%, such as 80% identity, such as at least 90% identity, such as at least 95% identity, such as at least 96% identity, such as at least 97% identity, such as at least 99% identity to the SEQ ID NO: 5 herein. In an embodiment the mature Bacillus stearothermophilus alpha-amylase, or variant thereof, is truncated, preferably to have around 485-496 amino acids, such as around 491 amino acids. Specific examples of alpha-amylases include the Bacillus amyloliquefaciens alpha-amylase of SEQ ID NO: 5 in WO 99/19467, the Bacillus licheniformis alpha-amylase of SEQ ID NO: 4 in WO 99/19467, and the Bacillus stearothermophilus alpha-amylase of SEQ ID NO: 3 in WO 99/19467. In an embodiment the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355. Specific alpha-amylase variants are disclosed in U.S. Pat. Nos. 6,093,562, 6,187,576, and 6,297,038 and include Bacillus stearothermophilus alpha-amylase (BSG alpha-amylase) variants having a deletion of one or two amino acids at positions R179 to G182, preferably a double deletion disclosed in WO 96/23873—see, e.g., page 20, lines 1-10, preferably corresponding to delta(181-182) compared to the amino acid sequence of Bacillus stearothermophilus alpha-amylase set forth in SEQ ID NO: 3 disclosed in WO 99/19467 or the deletion of amino acids R179 and G180 using SEQ ID NO: 3 in WO 99/19467 for numbering. In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be truncated so it has around 491 amino acids (compared to SEQ ID NO: 3 in WO 99/19467. Preferred are Bacillus alpha-amylases, especially Bacillus stearothermophilus alpha-amylases, which have a double deletion corresponding to a deletion of positions 181 and 182 and further comprise a N193F substitution (also denoted I181*+G182*+N193F) compared to the wild-type BSG alpha-amylase amino acid sequence set forth in SEQ ID NO: 3 disclosed in WO 99/19467. The bacterial alpha-amylase may also have a substitution in a position corresponding to S239 in the Bacillus licheniformis alpha-amylase shown in SEQ ID NO: 4 in WO 99/19467, or a S242 variant of the Bacillus stearothermophilus alpha-amylase of SEQ ID NO: 3 in WO 99/19467.

In a preferred embodiment the alpha-amylase is selected from the group of Bacillus stearomthermphilus alpha-amylase variants:

I181*+G182*+N193F+E129V+K177L+R179E;

I181*+G182*+N193F+V59A+Q89R+E129V+K177L+R179E+H208Y+K220P+N224L+Q254S;

I181*+G182*+N193F+V59A+Q89R+E129V+K177L+R179E+Q254S+M284V; and

I181*+G182*+N193F+E129V+K177L+R179E+K220P+N224L+S242Q+Q254S (using SEQ ID NO: 3 disclosed in WO 99/19467, or SEQ ID NO: 5 herein for numbering).

In another embodiment of the invention a glucoamylase may be added together with the protease or present and/or added during fermentation. The glucoamylase may be of microbial origin, e.g., the glucoamylase may be of fungal origin.

In one embodiment the glucoamylase is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, or a strain of Gloeophyllum, such as G. sepiarium or G. trabeum, or a strain of the Nigrofomes.

In an embodiment the glucoamylase is derived from Talaromyces, such as a strain of Talaromyces emersonii, such as the one disclosed in WO99/28448.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16).

In an embodiment the glucoamylase is derived from a strain of the genus Trametes, in particular a strain of Trametes cingulata disclosed in WO 2006/069289.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

In an embodiment of the process of the invention a desired fermentation product, such as especially ethanol, is produced from readily fermentable sugar-material by fermentation in a fermentation vat, the process comprises adding protease to the readily fermentable sugar material before feeding; feeding the protease-containing readily fermentable sugar material into the fermentation vat comprising the slurry of fermenting organisms; fermenting the readily fermentable sugar material into the desired fermentation product.

In a preferred embodiment ethanol is produced in a batch or fed-batch fermentation process in a fermentation vat comprising sugar cane molasses, comprising adding protease to the sugar cane molasses before feeding; feeding the protease-containing sugar cane molasses into the fermentation vat comprising a slurry of Saccharomyces cerevisae yeast; and fermenting the sugar cane molasses into ethanol.

In another embodiment a desired fermentation product, such as especially ethanol, is produced from readily fermentable sugar-material by fermentation in a fermentation vat, wherein the process comprises: feeding readily fermentable sugar material into the fermentation vat comprising a slurry of fermenting organisms; feeding protease into the fermentation vat comprising the slurry of readily fermentable sugars and fermenting organisms before fermentation; fermenting the readily fermentable sugar material into the desired fermentation product.

In a preferred embodiment ethanol is produced in a batch or fed-batch fermentation process in a fermentation vat comprising sugar cane molasses, wherein the process comprises: feeding sugar cane molasses into the fermentation vat comprising a slurry of Saccharomyces cerevisae yeast; feeding protease into the fermentation vat comprising the slurry of Saccharomyces cerevisae yeast and the sugar cane molasses before fermentation; fermenting the sugar cane molasses into ethanol.

In a further embodiment of the invention a desired fermentation product is produced from readily fermentable sugar material by fermentation in a fermentation vat, wherein the process comprises: feeding readily fermentable sugar-material into the fermentation vat comprising a slurry of fermenting organisms; adding protease into the fermentation vat during fermentation of the readily fermentable sugar material into the desired fermentation product.

In a preferred embodiment ethanol is produced as a batch or fed-batch fermentation process in a fermentation vat comprising sugar cane molasses, wherein the process comprises: feeding sugar cane molasses into the fermentation vat comprising a slurry of Saccharomyces cerevisae yeast; adding protease into the fermentation vat during fermentation of the sugar cane molasses into ethanol.

In a preferred specific embodiment the process of the invention, comprises

i) feeding the readily fermentable sugar material into the fermentation vat comprising a slurry of fermenting organism;

ii) fermenting the readily fermentable sugar material into a desired fermentation product,

wherein feeding of the readily fermentable sugar-material is done by introducing a feeding stream into the fermentation vat; wherein
S8A protease is mixed with the feeding stream before in step i); or
S8A protease is added to fermentation vat after feeding.

In a preferred embodiment the S8A protease is a S8A Thermococcus sp. protease preferably S8A Thermococcus litoralis protease, or S8A Thermococcus sp. PK protease.

The fermentation is done with a foaming fermenting organism, such as foaming yeast such as a foaming strain of the genus Saccharomyces, such as a foaming strain of Saccharomyces cerevisiae.

Use of Protease for Foam Reduction

In this aspect the invention relates to the use of S8A proteases for reducing foam generated by fermenting organisms when producing a desired fermentation product from readily fermentable sugars. In a preferred embodiment the desired fermentation product is produced according to a process of the invention.

The present invention is further described by the following numbered paragraphs:

Paragraph [1]. A process of producing a fermentation product from readily fermentable sugar-material in a fermentation vat comprising a fermentation medium using a fermenting organism, comprising:
i) feeding the readily fermentable sugar material into the fermentation vat comprising a slurry of fermenting organism;
ii) fermenting the readily fermentable sugar material into a desired fermentation product,
wherein a Thermococcus species S8A protease is added
a) before, during and/or after feeding in step i), and/or
b) during fermentation in step ii).
Paragraph [2]. The process of paragraph 1, wherein the readily fermentable sugar material is fed into the fermentation vat as a feeding stream.
Paragraph [3]. The process of paragraph 2, wherein the Thermococcus sp. S8A protease is mixed with the feeding stream of the readily fermentable sugar-material.
Paragraph [4]. The process of any of paragraphs 1-3, wherein the Thermococcus sp. S8A protease is mixed with the feeding stream of readily fermentable sugar-material before feeding step i).
Paragraph [5]. The process of any of paragraphs 1-4, wherein the readily fermentable sugars-material is selected from the group consisting of sugar cane juice, sugar cane molasses, sweet sorghum, sugar beets, and mixture thereof.
Paragraph [6]. The process of any one of paragraphs 1-5, wherein the fermenting organism is yeast, such as foaming yeast, e.g., from a strain of the genus Saccharomyces, such as a strain of Saccharomyces cerevisiae, especially a strain of Saccharomyces cerevisae generating foam when fermented.
Paragraph [7]. The process of any of paragraphs 1-6, wherein the fermenting organisms are recycled after fermentation in step ii).
Paragraph [8]. The process of any of paragraphs 1-7, wherein the fermenting organisms, such as foaming yeast, are collected after fermentation in step ii), acid washed, and recycled to the fermentation vat.
Paragraph [9]. The process of any of paragraphs 1-8, wherein the Thermococcus sp S8A protease is S8A Thermococcus litoralis protease, or S8A Thermococcus sp. PK protease.
Paragraph [10]. The process of any of paragraphs 1-9, wherein the S8A protease is selected from the group consisting of:
a) a polypeptide having at least 80%, at least 85, at least 90%, at least 91%, at least 92%, at least 93%, at least 94%, at least 95%, at least 96%, at least 97%, at least 98%, at least 99%, or 100% sequence identity to the mature polypeptides of SEQ ID NO: 2 or SEQ ID NO: 9;
b) a polypeptide encoded by a polynucleotide having at least 80%, at least 85, at least 90%, at least 91%, at least 92%, at least 93%, at least 94%, at least 95%, at least 96%, at least 97%, at least 98%, at least 99%, or 100% sequence identity to the mature polypeptide coding sequences of SEQ ID NO: 1 or SEQ ID NO: 8; or
c) a fragment of the polypeptides of (a), or (b) that has protease activity.
Paragraph [11]. The process of any of paragraphs 1-10, wherein the S8A protease has has at least 80%, at least 85, at least 90%, at least 91%, at least 92%, at least 93%, at least 94%, at least 95%, at least 96%, at least 97%, at least 98%, at least 99% or 100% sequence identity to the mature polypeptides of SEQ ID NO: 2 or SEQ ID NO: 9.
Paragraph [12]. The process of any of paragraphs 1-11, wherein the S8A proteases comprise or consist of SEQ ID NO: 2 or SEQ ID NO: 9 or the mature polypeptides of SEQ ID NO: 2 or SEQ ID NO: 9.
Paragraph [13]. The process of any of paragraphs 1-12, wherein the mature polypeptides are amino acids 107 to 424 of SEQ ID NO: 2 or amino acids 107 to 425 of SEQ ID NO: 9.
Paragraph [14]. The process of any of paragraphs 1-13, wherein the readily fermentable sugar-material substrate is not containing polysaccharide, such as starch and/or cellulose/hemicellulose.
Paragraph [15]. The process according to any of the preceding paragraphs, wherein the fermentation product is ethanol.
Paragraph [16]. The process of any of paragraphs 1-15, wherein the desired fermentation product is produced from readily fermentable sugar material by fermentation in a fermentation vat, the process comprises adding Thermococcus sp. S8A protease to the readily fermentable sugar material before feeding; feeding the protease-containing readily fermentable sugar material into the fermentation vat comprising a slurry of fermenting organisms; fermenting the readily fermentable sugar material into the desired fermentation product.
Paragraph [17]. The process of any of paragraphs 1-15, wherein ethanol is produced in a batch, fed batch, semi-continuous or continuous fermentation process in a fermentation vat comprising sugar cane molasses, comprising adding protease to the sugar cane molasses before feeding; feeding the Thermococcus sp. S8A protease-containing sugar cane molasses into the fermentation vat comprising a slurry of Saccharomyces cerevisae yeast; and fermenting the sugar cane molasses into ethanol.
Paragraph [18]. The process of any of paragraphs 1-15, wherein the desired fermentation product is produced from readily fermentable sugar material by fermentation in a fermentation vat, wherein the process comprises: feeding readily fermentable sugar material into the fermentation vat comprising a slurry of fermenting organisms; feeding Thermococcus sp. S8A protease into the fermentation vat comprising a slurry of readily fermentable sugars and fermenting organisms before fermentation; fermenting the readily fermentable sugar material into the desired fermentation product.
Paragraph [19]. The process of any of paragraphs 1-15, wherein ethanol is produced in a batch or fed batch fermentation process in a fermentation vat comprising sugar cane molasses, wherein the process comprises: feeding sugar cane molasses into the fermentation vat comprising a slurry of Saccharomyces cerevisae yeast; feeding Thermococcus sp. S8A protease into the fermentation vat comprising a slurry of Saccharomyces cerevisae yeast and the sugar cane molasses before fermentation; fermenting the sugar cane molasses into ethanol.
Paragraph [20]. The process of any of paragraphs 1-15, wherein the desired fermentation product is produced from readily fermentable sugar material by fermentation in a fermentation vat, wherein the process comprises: feeding readily fermentable sugar material into the fermentation vat comprising a slurry of fermenting organisms; adding Thermococcus sp. S8A protease into the fermentation vat during fermentation of the readily fermentable sugar-material into the desired fermentation product.
Paragraph [21]. The process of any of paragraphs 1-15, wherein ethanol is produced as a batch, fed batch, semi-continuous or continuous fermentation process in a fermentation vat comprising sugar cane molasses, wherein the process comprises: feeding sugar cane molasses into the fermentation vat comprising a slurry of Saccharomyces cerevisae yeast; adding Thermococcus sp. S8A protease, into the fermentation vat during fermentation of the sugar cane molasses into ethanol.
Paragraph [22]. The process of any of paragraphs 1-21, comprising:
i) feeding the readily fermentable sugar material into the fermentation vat comprising a slurry of fermenting organism;
ii) fermenting the readily fermentable sugar material into a desired fermentation product,
wherein feeding of the readily fermentable sugar-material is done by introducing a feeding stream into the fermentation vat; wherein
Thermococcus sp. S8A protease is mixed with the feeding stream before in step i); or
Thermococcus sp. S8A protease is added to fermentation vat after feeding.
Paragraph [23]. The process of paragraph 22, wherein the S8A protease is a S8A Thermococcus litoralis protease, or S8A Thermococcus sp. PK protease.
Paragraph [24]. Use of Thermococcus sp. S8A proteases for reducing foam generated by fermenting organisms when producing a desired fermentation product from readily fermentable sugars.
Paragraph [25]. The use according to paragraph 24, wherein the Thermococcus sp. S8A protease is selected from the group consisting of:
a) a polypeptide having at least 80%, at least 85, at least 90%, at least 91%, at least 92%, at least 93%, at least 94%, at least 95%, at least 96%, at least 97%, at least 98%, at least 99%, or 100% sequence identity to the mature polypeptides of SEQ ID NO: 2 or SEQ ID NO: 9;
b) a polypeptide encoded by a polynucleotide having at least 80%, at least 85, at least 90%, at least 91%, at least 92%, at least 93%, at least 94%, at least 95%, at least 96%, at least 97%, at least 98%, at least 99%, or 100% sequence identity to the mature polypeptide coding sequences of SEQ ID NO: 1 or SEQ ID NO: 8;
c) a fragment of the polypeptide of (a), or (b) that has protease activity.
Paragraph [26]. The use according to any of paragraphs 24-25, wherein the Thermococcus sp. S8A protease has at least 80%, at least 85, at least 90%, at least 91%, at least 92%, at least 93%, at least 94%, at least 95%, at least 96%, at least 97%, at least 98%, at least 99% or 100% sequence identity to the mature polypeptides of SEQ ID NO: 2 or SEQ ID NO: 9.
Paragraph [27]. The use according to any of paragraphs 24-26, wherein the mature polypeptides are amino acids 107 to 424 of SEQ ID NO: 2 or amino acids 107 to 425 of SEQ ID NO: 9.
Paragraph [28]. The use according to paragraph 24, wherein the S8A protease is a Thermococcus litoralis protease or a Thermococcus sp PK protease.
The present invention is described in further detail in the following examples which are offered to illustrate the present invention.

### EXAMPLES

### Strains

The Thermococcus strain 2319×1 was isolated from a hot spring located in the tidal zone near Goryachiy cape of Kunashir Island (South Kurils, Russian Far East region).

### Enzymes

### Protease Pfu:

Protease derived from Pyrococcus furiosus shown in SEQ ID NO: 7 herein.

### Yeast:

ETHANOL RED™ from Fermentis, USA

### Assays

### Protease Assays

### 1) Kinetic Suc-AAPF-pNA Assay:

pNA substrate: Suc-AAPF-pNA (Bachem L-1400).
Temperature: Room temperature (25° C.)
Assay buffers: 100 mM succinic acid, 100 mM HEPES, 100 mM CHES, 100 mM CABS, 1 mM CaCl₂, 150 mM KCl, 0.01% Triton X-100 adjusted to pH-values 2.0, 3.0, 4.0, 5.0, 6.0, 7.0, 8.0, 9.0, 10.0, and 11.0 with HCl or NaOH.

20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD₄₀₅ was monitored as a measure of the protease activity.

### 2) Endpoint Suc-AAPF-pNA AK Assay:

pNA substrate: Suc-AAPF-pNA (Bachem L-1400).
Temperature: controlled (assay temperature).
Assay buffer: 100 mM succinic acid, 100 mM HEPES, 100 mM CHES, 100 mM CABS, 1 mM CaCl₂, 150 mM KCl, 0.01% Triton X-100, pH 7.0.

200 μl pNA substrate (50 mg dissolved in 1.0 ml DMSO and further diluted 45× with the Assay buffer) were pipetted in an Eppendorf tube and placed on ice. 20 μl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate (1400 rpm.). The incubation was stopped by transferring the tube back to the ice bath and adding 600 μl 500 mM Succinic acid/NaOH, pH 3.5. After mixing the Eppendorf tube by vortexing 200 μl mixture was transferred to a microtiter plate. OD₄₀₅ was read as a measure of protease activity. A buffer blind was included in the assay (instead of enzyme).

The present invention is described in further detail in the following examples which are offered to illustrate the present invention.

### Example 1: Isolation of Thermococcus 2319×1

The organism was isolated from a hot spring located in the tidal zone near Goryachiy cape of Kunashir Island (South Kurils, Russian Far East region). An in situ enrichment was obtained in the Hungate tube containing birchwood xylan (Sigma) as a carbon source, amorphous Fe(III) oxide (ferrihydrite) as an electron acceptor, filled with a sample of sand and hot water from the spring and incubated for 6 days in the same spring, with temperature and pH fluctuating in the ranges of 76-99° C. and 5.0-7.0, respectively. The strain 2319×1 was isolated from this enrichment by 4 consequent transfers on a modified Pfennig medium with ferrihydrite (Slobodkin A. I., Reysenbach A.-L., Strutz N., Dreier M., Wiegel J. 1997. Thermoterrabacterium ferrireducens gen. nov., sp. nov. a thermophilic anaerobic, dissimilatory Fe(III)-reducing bacterium from a continental hot spring. Int. J. Syst. Bacteriol. V. 47. P. 541-547) containing 1 g/L birchwood xylan, 0.05 g/L yeast extract, 0.12 g/L Na₂S*9H₂O, 9 g/L NaCl, and 2 g/L MgCl₂*6H₂O, pH 6.8-7.0, incubated at 90° C.; at the final transfer ferrihydrite was substituted with elemental sulfur as the electron acceptor. Isolate grows optimally at 85° C., pH 6.9-7.0, 0.9% (m/v) NaCl, 10 g/L elemental sulfur. Among others, gelatine was to support growth of the strain. Cell yield during growth on gelatine was 1.5×10⁸ cells/mL. Protease(s) active against gelatine was detected by zymogram in suspension of whole cells grown with gelatine, in cell-free supernatant of this culture and in a fraction of cell surface proteins washed out with Tween 80. In all the fractions an active band of molecular weight >100 kDa was detected, in whole cell suspension and culture supernatant two different bands with lower molecular mass were also detected indicating possible multimeric structure of protease complex(es). According to the complete 16S rRNA gene sequence the isolate 2319×1 belongs to Thermococcus litoralis species (99% 16S rRNA gene identity with the type strain DSM 5473^{T} (NCBI blastn analysis with standard parameters excluding uncultured/environmental 16S rRNA sequences)).

### Example 2: Cloning and Expression of S8A Protease from Thermococcus 2319×1. Gene

The native gene of the Thermococcus S8A protease (SEQ ID NO: 1) was used as template for PCR amplification of the 1200 bp fragment corresponding to the predicted peptide of the Thermococcus S8A protease (amino acids 26-424 of SEQ ID NO: 2). The peptide of the Thermococcus S8A protease was fused to the Savinase secretion signal (with the following amino acid sequence: MKKPLGKIVASTALLISVAFSSSIASA disclosed as SEQ ID NO: 4) replacing the native secretion signal. The expressed DNA sequence was SEQ ID NO: 3.

### Expression Cloning

The 1200 bp fragment encoding the predicted mature peptide of the Thermococcus S8 protease was amplified by PCR and fused with regulatory elements and homology regions for recombination into the Bacillus subtilis genome. The linear integration construct was a SOE-PCR fusion product (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68) made by fusion of the gene between two Bacillus subtilis chromosomal regions along with strong promoters and a chloramphenicol resistance marker. The SOE PCR method is also described in patent application WO 2003095658.

The gene was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene was expressed with a Savinase secretion signal (encoding the following amino acid sequence: MKKPLGKIVASTALLISVAFSSSIASA) replacing the native secretion signal. The SOE-PCR product was transformed into Bacillus subtilis and integrated in the chromosome by homologous recombination into the pectate lyase locus. Subsequently a recombinant Bacillus subtilis clone containing the integrated expression construct was grown in liquid culture. The culture broth was centrifuged (20000×g, 20 min) and the supernatant was carefully decanted from the precipitate and used for purification of the enzyme disclosed herein as SEQ ID NO: 2.

### Example 3: Purification of the S8A Protease from Thermococcus litoralis (SEQ ID NO: 2)

The culture broth was centrifuged (20000×g, 20 min) and the supernatant was carefully decanted from the precipitate. The supernatant was filtered through a Nalgene 0.2 μm filtration unit in order to remove the rest of the Bacillus host cells. The 0.2 μm filtrate was transferred to 10 mM Tris/HCl, 1 mM CaCl₂, pH 9.0 on a G25 Sephadex column (from GE Healthcare) and the G25 transferred enzyme was applied to a Q Sepharose FF column (from GE Healthcare) equilibrated in 10 mM Tris/HCl, 1 mM CaCl₂, pH 9.0. After washing the column extensively with the equilibration buffer, the protease was eluted with a linear gradient between the equilibration buffer and 10 mM Tris/HCl, 1 mM CaCl₂, 1.0M NaCl, pH 9.0 over five column volumes. Fractions from the column were analysed for protease activity (using the Kinetic Suc-AAPF-pNA assay at pH 9) and the major activity peak was pooled. The pool from the Q Sepharose column was diluted 8× with deionized water and the pH of the diluted pool was adjusted to pH 6.0 with 20% CH₃COOH. The adjusted pool was applied to a Bacitracin agarose column (from UpFront chromatography) equilibrated in 100 mM H₃BO₃, 10 mM MES, 2 mM CaCl₂, pH 6.0. After washing the column extensively with the equilibration buffer, the protease was eluted with 100 mM H₃BO₃, 10 mM MES, 2 mM CaCl₂, 1.0M NaCl, pH 6.0+25% (v/v) isopropanol. The eluted peak was transferred to 10 mM Tris/HCl, 1 mM CaCl₂, pH 9.0 on a G25 Sephadex column (from GE Healthcare) and the buffer transferred enzyme was applied to a SOURCE Q column (from GE Healthcare) equilibrated in 10 mM Tris/HCl, 1 mM CaCl₂, pH 9.0. After washing the column extensively with the equilibration buffer, the protease was eluted with a linear gradient between the equilibration buffer and 10 mM Tris/HCl, 1 mM CaCl₂, 1.0M NaCl, pH 9.0 over five column volumes. Fractions from the column were analysed for protease activity (using the Kinetic Suc-AAPF-pNA assay at pH 9) and fractions with activity were analysed by SDS-PAGE. Fractions where only one band was seen on the Coomassie stained gel were pooled and pH was adjusted to pH 7.0 with 0.5M HCl. The pH adjusted pool was the purified preparation and was used for further characterization. The polypeptide shown as amino acids 107 to 424 of SEQ ID NO: 2 showed protease activity as shown below.

### Example 4: Cloning and Expression of S8A Protease from Thermococcus sp. PK. Gene

The Thermococcus sp. PK S8A protease was expressed from a synthetic gene in Bacillus subtilis. The synthetic gene sequence was designed based on peptide sequence of the NCBI Reference Sequence WP_042702525.1 enclosed herein as SEQ ID NO: 9 and codon optimized for expression in Bacillus subtilis. The peptide of the Thermococcus sp. PK S8A protease was expressed with a Savinase secretion signal (with the following amino acid sequence: MKKPLGKIVASTALLISVAFSSSIASA disclosed as SEQ ID NO: 4) replacing the native secretion signal. The expressed DNA sequence was SEQ ID NO: 10.

### Expression Cloning

The 1200 bp fragment corresponding to the predicted mature peptide of the Thermococcus S8A protease was PCR amplified from the standard cloning vector containing the synthetic gene. The PCR primers were designed with 15 bp extensions (5′) complementary to the ends of the linearized vector. A ClaI restriction site was incorporated into 5′ extension of the forward primer and a MluI restriction site in the 5′ extension of the reverse primer to facilitate use of the IN-FUSION™ Cloning Kit (BD Biosciences, Palo Alto, Calif., USA) to clone the fragment directly into the expression vector ExpVec8. The expression vector, Expvec8 was digested with the same restriction enzymes (ClaI and MluI). The cloning protocol was performed according to the IN-FUSION™ Cloning Kit instructions. The treated plasmid and insert were transformed into One Shot® TOP10F′ Chemically Competent E. coli cells (Invitrogen, Carlsbad, Calif., USA) according to the manufacturer's protocol. Integration of the insert into the vector and nucleotide sequence of the insert was verified by sequencing of isolated plasmids. A representative plasmid expression clone that was free of PCR errors was transformed into Bacillus subtilis. A recombinant Bacillus subtilis clone containing the integrated expression construct were grown in liquid culture. The culture broth was centrifuged (20000×g, 20 min) and the supernatant was carefully decanted from the precipitate and used for purification of the enzyme disclosed herein as SEQ ID NO: 9.

### Example 5: Purification of the S8A Protease from Thermococcus sp. PK (SEQ ID NO: 9)

The culture broth is centrifuged (20000×g, 20 min) and the supernatant is carefully decanted from the precipitate. The supernatant is filtered through a Nalgene 0.2 μm filtration unit in order to remove the rest of the Bacillus host cells. The 0.2 μm filtrate was transferred to 10 mM Tris/HCl, 1 mM CaCl₂, pH 9.0 on a G25 Sephadex column (from GE Healthcare) and the G25 transferred enzyme was applied to a Q Sepharose FF column (from GE Healthcare) equilibrated in 10 mM Tris/HCl, 1 mM CaCl₂, pH 9.0. After washing the column extensively with the equilibration buffer, the protease is eluted with a linear gradient between the equilibration buffer and 10 mM Tris/HCl, 1 mM CaCl₂, 1.0M NaCl, pH 9.0 over five column volumes. Fractions from the column are analysed for protease activity (using the Kinetic Suc-AAPF-pNA assay at pH 9) and the major activity peak is pooled. The pool from the Q Sepharose column is diluted 8× with deionized water and the pH of the diluted pool is adjusted to pH 6.0 with 20% CH₃COOH. The adjusted pool is applied to a Bacitracin agarose column (from UpFront chromatography) equilibrated in 100 mM H₃BO₃, 10 mM MES, 2 mM CaCl₂, pH 6.0. After washing the column extensively with the equilibration buffer, the protease is eluted with 100 mM H₃BO₃, 10 mM MES, 2 mM CaCl₂, 1.0M NaCl, pH 6.0+25% (v/v) isopropanol. The eluted peak was transferred to 10 mM Tris/HCl, 1 mM CaCl₂, pH 9.0 on a G25 Sephadex column (from GE Healthcare) and the buffer transferred enzyme was applied to a SOURCE Q column (from GE Healthcare) equilibrated in 10 mM Tris/HCl, 1 mM CaCl₂, pH 9.0. After washing the column extensively with the equilibration buffer, the protease is eluted with a linear gradient between the equilibration buffer and 10 mM Tris/HCl, 1 mM CaCl₂, 1.0M NaCl, pH 9.0 over five column volumes. Fractions from the column are analysed for protease activity (using the Kinetic Suc-AAPF-pNA assay at pH 9) and fractions with activity are analysed by SDS-PAGE. Fractions where only one band is seen on the Coomassie stained gel are pooled and pH is adjusted to pH 7.0 with 0.5M HCl. The pH adjusted pool is the purified preparation and is used for further characterization. The mature polypeptide of SEQ ID NO: 9 is tested for protease activity as shown in example 7 below.

### Example 6: Characterization of the S8A Protease from Thermococcus litoralis (SEQ ID NO: 2)

The kinetic Suc-AAPF-pNA assay was used for obtaining the pH-activity profile and the pH-stability profile for the S8A protease from Thermococcus sp. For the pH-stability profile the protease was diluted 10× in the different Assay buffers to reach the pH-values of these buffers and then incubated for 2 hours at 37° C. After incubation, the pH of the protease incubations was transferred to pH 8.0, before assay for residual activity, by dilution in the pH 8.0 Assay buffer. The endpoint Suc-AAPF-pNA assay was used for obtaining the temperature-activity profile at pH 7.0.

The results are shown in Tables 1-3 below. For Table 1, the activities are relative to the optimal pH for the enzyme. For Table 2, the activities are residual activities relative to a sample, which were kept at stable conditions (5° C., pH 8.0). For Table 3, the activities are relative to the optimal temperature for the enzyme at pH 7.0.

TABLE 1
pH-activity profile

| | S8A Protease from Thermococcus sp. |
|---|---|
| pH | SEQ ID NO: 2 |
| 2 | 0.00 |
| 3 | 0.00 |
| 4 | 0.00 |
| 5 | 0.02 |
| 6 | 0.14 |
| 7 | 0.60 |
| 8 | 1.00 |
| 9 | 0.95 |
| 10 | 0.60 |
| 11 | 0.16 |

TABLE 2
pH-stability profile (residual activity after 2 hours at 37° C.)

| pH | S8A Protease from Thermococcus sp. |
|---|---|
| 2 | 0.00 |
| 3 | 0.72 |
| 4 | 0.96 |
| 5 | 0.99 |
| 6 | 1.00 |
| 7 | 1.00 |
| 8 | 1.01 |
| 9 | 1.02 |
| 10 | 0.98 |
| 11 | 0.99 |
| After 2 hours at 5° C. | 1.00 (at pH 8) |

TABLE 3
Temperature activity profile at pH 7.0

| Temp (° C.) | S8A Protease from Thermococcus sp. (pH 8) |
|---|---|
| 15 | 0.18 |
| 25 | 0.38 |
| 37 | 0.63 |
| 50 | 0.93 |
| 60 | 1.00 |
| 70 | 0.99 |
| 80 | 0.90 |
| 90 | 0.77 |

### Example 7: Determination of the N-Terminal of Mature Polypeptide

The mature sequence, based on EDMAN N-terminal sequencing data and Intact MS data was determined to be amino acids 107-424 of SEQ ID NO: 2.

The calculated molecular weight from this mature sequence was 32966.1 Da.

The relative molecular weight as determined by SDS-PAGE was approx. Mᵣ=37 kDa.

The molecular weight determined by Intact molecular weight analysis was 32965.4 Da.

### Example 8: Characterization of the S8A Protease from Thermococcus sp. PK (SEQ ID NO: 9)

The kinetic Suc-AAPF-pNA assay is used for obtaining the pH-activity profile and the pH-stability profile for the S8A Protease from Thermococcus sp PK. For the pH-stability profile the protease is diluted 10× in the different Assay buffers to reach the pH-values of these buffers and then incubated for 2 hours at 37° C. After incubation, the pH of the protease incubations is transferred to pH 8.0, before assay for residual activity, by dilution in the pH 8.0 Assay buffer. The endpoint Suc-AAPF-pNA assay is used for obtaining the temperature-activity profile at pH 7.0.

The results are shown in Tables 4-6 below. For Table 4, the activities are relative to the optimal pH for the enzyme. For Table 5, the activities are residual activities relative to a sample, which were kept at stable conditions (5° C., pH 8.0). For Table 6, the activities are relative to the optimal temperature for the enzyme at pH 7.0.

TABLE 4
pH-activity profile

| | S8 Protease from Thermoccus sp. PK |
|---|---|
| pH | SEQ ID NO: 9 |
| 2 | 0.00 |
| 3 | 0.00 |
| 4 | 0.00 |
| 5 | 0.03 |
| 6 | 0.19 |
| 7 | 0.63 |
| 8 | 0.94 |
| 9 | 1.00 |
| 10 | 0.67 |
| 11 | 0.05 |

TABLE 5
pH-stability profile (residual activity after 2 hours at 37° C.)

| | S8 Protease from Thermoccus sp. |
|---|---|
| pH | PK (SEQ ID NO: 9) |
| 2 | 0.00 |
| 3 | 0.85 |
| 4 | 1.13 |
| 5 | 1.07 |
| 6 | 1.05 |
| 7 | 1.05 |
| 8 | 1.04 |
| 9 | 1.02 |
| 10 | 1.02 |
| 11 | 1.02 |
| After 2 | 1.00 |
| hours at | (at pH 8) |
| 5° C. | |

TABLE 6
Temperature activity profile at pH 7.0

| | S8 Protease from Thermoccus sp. PK (pH 7) |
|---|---|
| Temp (° C.) | (SEQ ID NO: 9) |
| 15 | 0.11 |
| 25 | 0.22 |
| 37 | 0.44 |
| 50 | 0.70 |
| 60 | 0.88 |
| 70 | 1.00 |
| 80 | 0.97 |
| 90 | 0.99 |
| 99 | 0.84 |

Other Characteristics for the S8 Protease (SEQ ID NO: 9) from Thermococcus sp. PK Inhibitor: PMSF.

The relative molecular weight as determined by SDS-PAGE was approx. Mᵣ=37 kDa.

The observed molecular weight determined by Intact molecular weight analysis for a PMSF treated sample was 33089.2 Da. PMSF adds 154.2 Da to the mass and hence the observed mass for the protease part is 32935.0 Da.

The mature polypeptide sequence (from EDMAN N-terminal sequencing data and Intact MS data): Amino acids 107-425 of SEQ ID NO: 9.

The calculated molecular weight from this mature sequence was 32934.9 Da.

### Example 9: Comparison Between S8A Thermococcus litoralis Protease (Mature Polypeptide of SEQ ID NO: 2) and PfuS Protease (SEQ ID NO: 7) on Foam Control in Sugarcane Molasses Fermentation

S. cerevisiae stock cultures were grown in shake flasks containing YPD medium (1% yeast extract, 2% bacteriological peptone, 2% dextrose). After overnight growth, 20% (v/v) glycerol was added and 1 mL aliquots were stored at −80° C. Stock cultures were used to prepare pre-cultures for fermentation trials experiments.

### Fermentation Must Preparation

The musts used for the fermentation experiments were prepared by diluting sugarcane molasses (commercially available) to obtain a sufficient amount to feed every tube. This was done every day and the remaining diluted molasses was discarded.

### Fermentation Trials

Yeast cells were plated on YPD-agar medium and incubated for 48 h at 30° C. A single cell isolate was transferred to 5 mL liquid YPD and incubated overnight at 30° C. The whole content was transferred to sterile molasses medium diluted to 10% (w/v) total sugars (sucrose, glucose and fructose expressed as hexose content) supplemented with 5 g/L yeast extract, and incubated for 48 h at 30° C. Yeast biomass was collected by centrifugation (4000 rpm for 10 min) for fermentation trials.

Fermentation trials were performed at 32° C. in 50 mL centrifuge vials (TPP), simulating as far as possible the industrial fermentation process as performed in Brazil. A fermentation substrate containing 20° Brix (composed of diluted molasses) was fed into the yeast slurry. The yeast slurry represented 30% of the total fermentation volume, similar to industrial conditions. After fermentation, yeast cells were collected by centrifugation (4000 rpm for 10 min), weighed, diluted with fermented must and water (to 35% w/v yeast wet weight), and treated with sulfuric acid (pH from 2.5 for 1 h) and reused in a subsequent fermentation cycle, comprising 8 fermentation cycles. Samples were run in triplicate for each condition.

### Determination of Biomass

Wet weight biomass was determined gravimetrically after centrifugation (4000 rpm for 10 min) of the samples.

### Foam Measurement

S8A protease (amino acids 107-424 of SEQ ID NO: 2), which is an acidic protease, was evaluated whether it can withstand the conditions of a sugarcane molasses fermentation. The performance of the Thermococcus litoralis S8A protease for foam control during sugarcane molasses fermentation was compared to the Pfus protease.

The fermentation experiment was performed in 8 fermentation cycles according to the Material and Methods section. Each cycle represented a turn of 1) yeast slurry preparation (35% w/w) using fermented must and water (1:1); 2) addition of H₂SO₄ to pH 2.5 for 1 h at room temperature; 3) and feeding with diluted molasses (20° Brix) to result in cell density of 10% (w/w) followed by incubation at 32° C. for 7-9 h. Addition of 5 ppm (mg/L) of enzyme (at the feeding molasses) started from the 2^{nd} cycle onwards. The data about the enzymes added are presented in Table 7. During the study, enzyme was added during 7 cycles of fermentation.

TABLE 7
Proteases tested for foam control
in sugarcane molasses fermentation.

| | | Concentration | |
|---|---|---|---|
| Donor organism | Family | (mg/mL) | pH optimum |
| Pyrococcus | S8 | 10.05 | pH 11 |
| furiosus, PfuS | | | |
| Thermococcus sp | S8 | 2.67 | pH 8.5 |

Foam was registered every hour after feeding for each cycle by recording the foam height in tubes and/or by taking pictures of representative tubes.

The calculation of foam height was done by dividing the total volume in the tube (foam+liquid) by the liquid volume. Usually, fermentations in Brazil are performed leaving a 30% total vat volume as a headspace for foam formation. Only when foam reaches the top of the vessel, antifoams are added. Therefore, keeping foam bellow this threshold limit is considered foam control for the industry. In our laboratory assays 100% foam volume indicates that foam is in the same level of fermentation broth, or no foam formation. In order to indicate a foam formation, as done in industry, foam should rise above 143% in lab scale assays.

From the results, it was observed that S8A protease showed a similar performance to the PfuS. Foam measurements resulted in the following data, shown in Table 8.

TABLE 8
Foam control measured as foam height (%).

| | | Fermentation Time (h) | | | | | |
|---|---|---|---|---|---|---|---|
| | | 1:00 | 2:00 | 3:00 | 4:00 | 5:00 | 6:00 |
| Cycle 1(1A) | Control | 189 | 263 | 159 | 149 | 167 | 140 |
| | PfuS | 217 | 263 | 150 | 166 | 174 | 156 |
| | SEQ ID NO: 7 | | | | | | |
| | SEQ ID NO: 2 | 179 | 259 | 146 | 150 | 153 | 153 |
| Cycle 2(1B) | Control | 252 | 199 | 190 | 194 | 151 | 164 |
| | PfuS | 235 | 246 | 150 | 129 | 100 | 100 |
| | SEQ ID NO: 2 | 271 | 144 | 138 | 128 | 124 | 117 |
| Cycle 3(1C) | Control | 243 | 165 | 154 | 157 | 150 | 161 |
| | PfuS | 118 | 100 | 100 | 105 | 100 | 100 |
| | SEQ ID NO: 2 | 151 | 142 | 110 | 119 | 110 | 100 |
| Cycle 4(1D) | Control | 224 | 150 | 159 | 174 | 137 | 132 |
| | PfuS | 114 | 135 | 152 | 159 | 100 | 100 |
| | SEQ ID NO: 2 | 108 | 140 | 145 | 145 | 121 | 128 |
| Cycle 5(1E) | Control | 231 | 152 | 161 | 133 | 142 | 132 |
| | PfuS | 120 | 132 | 155 | 104 | 104 | 100 |
| | SEQ ID NO: 2 | 123 | 140 | 162 | 128 | 103 | 100 |
| Cycle 6(1F) | Control | 194 | 232 | 170 | 160 | 163 | 119 |
| | PfuS | 100 | 119 | 145 | 108 | 100 | 100 |
| | SEQ ID NO: 2 | 100 | 129 | 150 | 120 | 110 | 100 |
| Cycle 7(1G) | Control | 179 | 141 | 153 | 148 | 142 | 130 |
| | PfuS | 100 | 138 | 147 | 133 | 100 | 100 |
| | SEQ ID NO: 2 | 100 | 141 | 145 | 151 | 100 | 114 |
| Cycle 8(1H) | Control | 179 | 140 | 135 | 144 | 142 | 139 |
| | PfuS | 100 | 131 | 128 | 130 | 110 | 100 |
| | SEQ ID NO: 2 | 100 | 131 | 135 | 115 | 107 | 107 |

### Example 10: Comparison Between S8A Thermococcus sp. PK Protease (Mature Polypeptide of SEQ ID NO: 9) and Mg Prot III (SEQ ID NO: 11) on Foam Control in Sugarcane Molasses Fermentation

S. cerevisiae stock cultures were grown in shake flasks containing YPD medium (1% yeast extract, 2% bacteriological peptone, 2% dextrose). After overnight growth, 20% (v/v) glycerol was added and 1 mL aliquots were stored at −80° C. Stock cultures were used to prepare pre-cultures for fermentation trials experiments.

### Fermentation Must Preparation

The musts used for the fermentation experiments were prepared by diluting sugarcane molasses (commercially available) to obtain a sufficient amount to feed every tube. This was done every day and the remaining diluted molasses was discarded.

### Fermentation Trials

Yeast cells were plated on YPD-agar medium and incubated for 48 h at 30° C. A single cell isolate was transferred to 5 mL liquid YPD and incubated overnight at 30° C. The whole content was transferred to sterile molasses medium diluted to 10% (w/v) total sugars (sucrose, glucose and fructose expressed as hexose content) supplemented with 5 g/L yeast extract, and incubated for 48 h at 30° C. Yeast biomass was collected by centrifugation (4000 rpm for 10 min) for fermentation trials.

Fermentation trials were performed at 32° C. in 50 mL centrifuge vials (TPP), simulating as far as possible the industrial fermentation process as performed in Brazil. A fermentation substrate containing 20° Brix (composed of diluted molasses) was fed into the yeast slurry. The yeast slurry represented 30% of the total fermentation volume, similar to industrial conditions. After fermentation, yeast cells were collected by centrifugation (4000 rpm for 10 min), weighed, diluted with fermented must and water (to 35% w/v yeast wet weight), and treated with sulfuric acid (pH from 2.5 for 1 h) and reused in a subsequent fermentation cycle, comprising 8 fermentation cycles. Samples were run in triplicate for each condition.

### Determination of Biomass

Wet weight biomass was determined gravimetrically after centrifugation (4000 rpm for 10 min) of the samples.

### Foam Measurement

S8A protease (amino acids 107-425 of SEQ ID NO: 9), which is an acidic protease, was evaluated whether it can withstand the conditions of a sugarcane molasses fermentation. The performance of the Thermococcus sp. PK S8A protease for foam control during sugarcane molasses fermentation was compared to Meripilus giganteus serine protease (Mg Prot III) previously disclosed in WO 2014/037438, and included herein as SEQ ID NO: 11. The fermentation experiment was performed in 8 fermentation cycles according to the Material and Methods section. Each cycle represented a turn of 1) yeast slurry preparation (35% w/w) using fermented must and water (1:1); 2) addition of H₂SO₄ to pH 2.5 for 1 h at room temperature; 3) and feeding with diluted molasses (20° Brix) to result in cell density of 10% (w/w) followed by incubation at 32° C. for 7-9 h. Addition of 1 ppm (mg/L) of enzyme (at the feeding molasses) started from the 2^{nd} cycle onwards. The data about the enzymes added are presented in Table 9. During the study, enzyme was added during 7 cycles of fermentation.

TABLE 9
Proteases tested for foam control in sugarcane molasses fermentation.

| Donor | | Concentration | |
|---|---|---|---|
| organism | Family | (mg/mL) | pH optimum |
| MG Protease III | S53 | 10.05 | pH 11 |
| SEQ ID NO: 11 | | | |
| Thermococcus | S8 | 0.59 | pH 8.5 |
| sp PK | | | |
| SEQ ID NO: 9 | | | |

Foam was registered every hour after feeding for each cycle by recording the foam height in tubes from cycle 4 onwards, and/or by taking pictures of representative tubes.

The calculation of foam height was done by dividing the total volume in the tube (foam+liquid) by the liquid volume. Usually, fermentations in Brazil are performed leaving a 30% total vat volume as a headspace for foam formation. Only when foam reaches the top of the vessel, antifoams are added. Therefore, keeping foam bellow this threshold limit is considered foam control for the industry. In our laboratory assays 100% foam volume indicates that foam is in the same level of fermentation broth, or no foam formation. In order to indicate a foam formation, as done in industry, foam should rise above 143% in lab scale assays.

From the results, it was observed that S8A protease showed a similar performance to the Mg Prot III. Foam measurements resulted in the following data, shown in Table 10.

TABLE 10
Foam control measured as foam height (%).

| | | Fermentation Time (h) | | | | | |
|---|---|---|---|---|---|---|---|
| | | 1:00 | 2:00 | 3:00 | 4:00 | 5:00 | 6:00 |
| Cycle 4(1D) | Control | 179 | 167 | 151 | 137 | 139 | ND |
| | Mg Prot III | 148 | 163 | 154 | 144 | 128 | ND |
| | SEQ ID NO: 9 | 148 | 152 | 151 | 131 | 124 | ND |
| Cycle 5(1E) | Control | 186 | 164 | 163 | 150 | 139 | ND |
| | Mg Prot III | 119 | 178 | 157 | 142 | 138 | ND |
| | SEQ ID NO: 9 | 123 | 166 | 147 | 143 | 132 | ND |
| Cycle 6(1F) | Control | 216 | 222 | 157 | 138 | 130 | ND |
| | Mg Prot III | 161 | 167 | 136 | 136 | 129 | ND |
| | SEQ ID NO: 9 | 163 | 148 | 140 | 132 | 126 | ND |
| Cycle 7(1G) | Control | 203 | 163 | 162 | 142 | 141 | ND |
| | Mg Prot III | 156 | 187 | 141 | 140 | 133 | ND |
| | SEQ ID NO: 9 | 164 | 137 | 140 | 133 | 126 | ND |
| Cycle 8(1H) | Control | 186 | 183 | 179 | 167 | 131 | 136 |
| | Mg Prot III | 155 | 200 | 167 | 157 | 123 | 124 |
| | SEQ ID NO: 9 | 163 | 167 | 154 | 154 | 124 | 126 |

1: A process of producing a fermentation product from readily fermentable sugar-material in a fermentation vat comprising a fermentation medium using a fermenting organism, the process comprising:
i) feeding the readily fermentable sugar-material into the fermentation vat comprising a slurry of fermenting organism;
ii) fermenting the readily fermentable sugar-material into a desired fermentation product,
wherein a Thermococcus species S8A protease is added
a) before, during and/or after feeding in step i), and/or
b) during fermentation in step ii).

2: The process of claim 1, wherein the readily fermentable sugar-material is selected from the group consisting of sugar cane juice, sugar cane molasses, sweet sorghum, sugar beets, and mixture thereof.

3: The process of claim 1, wherein the fermenting organism is yeast.

4: The process of claim 1, wherein the Thermococcus sp. S8A protease is a Thermococcus litoralis protease, or a Thermococcus sp. PK protease.

5: The process of claim 1, wherein the Thermococcus sp. S8A protease is selected from the group consisting of:
(a) a polypeptide having at least 80% sequence identity to the mature polypeptide of SEQ ID NO: 2 or SEQ ID NO: 9;
(b) a polypeptide encoded by a polynucleotide having at least 80% sequence identity to the mature polypeptide coding sequence of SEQ ID NO: 1 or SEQ ID NO: 8;
(c) a fragment of the polypeptide of (a), or (b) that has protease activity.

6: The process of claim 1, wherein the Thermococcus sp. S8A protease comprises or consists of SEQ ID NO: 2 or SEQ ID NO: 9 or the mature polypeptide of SEQ ID NO: 2 or SEQ ID NO: 9.

7: The process of claim 1, wherein the mature polypeptide of SEQ ID NO: 2 is amino acids 107 to 424 of SEQ ID NO: 2, and wherein the mature polypeptide of SEQ ID NO: 9 is amino acids 107 to 425 of SEQ ID NO: 9.

8: The process of claim 1, wherein the readily fermentable sugar-material substrate does not contain polysaccharide.

9: The process according to claim 1, wherein the fermentation product is ethanol.

10-14. (canceled)

15: The process of claim 1, wherein the fermenting organism generates foam when fermented without the presence of Thermococcus species S8A protease.

16: The process of claim 1, wherein the fermenting organism is a strain of Saccharomyces cerevisiae.

17: The process of claim 1, wherein the Thermococcus sp. S8A protease is mixed with the feeding stream of readily fermentable sugar-material before feeding step i).

18: The process of claim 1, wherein the fermenting organism is recycled after fermentation in step ii).

19: The process of claim 1, wherein feeding of the readily fermentable sugar-material of step i) is done by introducing a feeding stream into the fermentation vat; and
wherein the Thermococcus sp. S8A protease is mixed with the feeding stream before feeding step i), or the Thermococcus sp. S8A protease is added to fermentation vat after feeding step i).
